(19) United States
(12) Patent Application Publication
(10) Pub. No.: US 2013/0039951 A1
(43) Pub. Date: Feb. 14, 2013

(54) Process Of Preparing A Stabilized And Solubilized Formulation Of Sirolimus Derivatives

(71) Applicants: Soon-Hoe Kim, Gyeonggi-do (KR); Mi-Won Son, Gyeonggi-do (KR); Sun-Woo Jang, Seoul (KR); Sang-Kuk Park, Gyeonggi-do (KR); Sang-Dug Han, Gyeonggi-do (KR); Myung-Joo Kang, Gyeonggi-do (KR); Kyung-Wan Ma, Gyeonggi-do (KR)

(72) Inventors: Soon-Hoe Kim, Gyeonggi-do (KR); Mi-Won Son, Gyeonggi-do (KR); Sun-Woo Jang, Seoul (KR); Sang-Kuk Park, Gyeonggi-do (KR); Sang-Dug Han, Gyeonggi-do (KR); Myung-Joo Kang, Gyeonggi-do (KR); Kyung-Wan Ma, Gyeonggi-do (KR)

(73) Assignee: DONG-A PHARM. CO., LTD., Seoul (KR)

(21) Appl. No.: 13/249,513

(22) Filed: Sep. 30, 2011

(30) Foreign Application Priority Data
Aug. 11, 2011 (KR) 10-2011-0080326

(51) Int. Cl. A61K 31/439; A61K 9/14; A61P 37/06; A61K 31/695

(52) U.S. Cl. 424/400; 514/291; 514/63

(57) ABSTRACT

Provided is a process for preparing a solubilized and stabilized formulation of a sirolimus derivative, which comprises the steps of a dissolving a sirolimus derivative in a solvent, and bring a solution of the sirolimus derivative into contact with a water-soluble carrier to disperse the sirolimus derivative in the water-soluble carrier, and a formulation of a sirolimus derivative with improved solubility and stability as prepared by the preparation process as above.

### CROSS-REFERENCE TO RELATED APPLICATION

This application claims priority to and the benefit of Korean Patent Application No. 10-2011-0080326, filed in Korean Intellectual Property Office on Aug. 11, 2011, the entire contents of which are incorporated herein by reference.

### BACKGROUND OF THE INVENTION

(a) Field of the Invention

The present invention relates to a process for preparing a stabilized and solubilized formulation of sirolimus derivatives, comprising the steps of dissolving a sirolimus derivative in a solvent, and contacting the solution of the sirolimus derivative with a water-soluble carrier, to disperse the sirolimus derivative in the water-soluble carrier; and a sirolimus derivative formulation prepared thereby with enhanced solubility and stability.

(b) Description of the Related Art

Sirolimus, also known as rapamycin, is a macrolide discovered in a type of bacteria, Streptomyces hygroscopicus, and is a drug used to prevent rejection in organ transplantation and marketed under the trade name Rapamune®. Sirolimus binds the cytosolic protein, FK-binding protein 12 (FKBP12) and the sirolimus-FKBP12 complex as produced directly binds the mTOR Complex1 (mTORC1) to inhibit the mammalian target of rapamycin (mTOR) pathway, thereby blocking activation of T- and B-cells and showing a pharmacological action.

Everolimus is a derivative of sirolimus wherein a hydroxyethyl group is added to the 40-O-silolimus, and is marketed by Novartis under the trade names Zortress (in the US) and Certican (in Europe and Republic of Korea) as a medicine for preventing rejection in organ transplantation. Besides the use as an immunosuppressant, this drug inhibits mTOR pathway to inhibit expression of vascular endothelial growth factor (VEGF), thereby exhibiting an anticancer activity. Thus, it is recently marketed under the trade name of Afinitor® for the purpose of treating advanced renal cell carcinoma, which has been failed to be treated by Sunitinib or Sorafenib. Many clinical trials have been under way in breast cancer, gastric cancer, hepatoma, pancreatic cancer, and the like.

Besides the foregoing, many derivatives of sirolimus were known in the art. Certain 16-O-substituted sirolimus derivatives were disclosed in WO 94/02136. 40-O-substituted sirolimus derivatives were also disclosed, for example, in U.S. Pat. No. 5,258,389 and WO94/09010 (O-aryl and O-alkyl rapamycins); WO92/05179 (carboxylic acid esters), U.S. Pat. No. 5,118,677 (amide esters), U.S. Pat. No. 5,118,678 (carbamates), U.S. Pat. No. 5,100,883 (fluorinated esters), U.S. Pat. No. 5,151,413 (acetals), U.S. Pat. No. 5,120,842 (silyl ethers), WO 93/11130 (methylene rapamycins and their derivatives), WO 94/02136 (methoxy derivatives), WO 94/02385 and WO 95/14023 (alkenyl derivatives), all of which are incorporated herein by reference. 32-O-dihydro or substituted sirolimus derivatives were disclosed in U.S. Pat. No. 5,256,790. Other sirolimus derivatives include the ones as described in PCT/EP96/02441 such as 32-deoxorapamycin, 16-pent-2-ynyloxy-32(S)-dihydrorapamycin, and the like. Sirolimus, its structurally similar homologous, and its derivatives are termed collectively herein as “sirolimus derivatives.”

On oral administration to human, a solid sirolimus derivative such as everolimus has a low water solubility and a high molecular weight such that they have difficulties in permeating a gastrointestinal membrane. In addition, it serves as an efflux pump substrate such as P-glycoprotein so that it may not absorbed to any significant extent into the blood stream. Simple mixtures of the sirolimus derivatives including everolimus with typical pharmaceutical excipients were known in the art, but they found to have drawbacks such as an unpredictable dissolution rate, non-uniform bioavailability, and instability.

Korean Patent No. 0352943 disclosed a pharmaceutical composition for oral administration in the form of a co-precipitated solid dispersion comprising a sirolimus derivative and a carrier. Although such inventive composition made an improvement to have a higher dissolution rate, it was prepared through the extremely complex process composed of the steps of mixing and dissolving a sirolimus derivative and a carrier in an organic solvent, evaporating the solvent, pulverizing dry residues thus obtained into particulates having an average diameter of less than 0.5 mm, mixing the particulates with typical pharmaceutical excipients, and tableting the resulting mixture. Not only does such complex and discontinuous preparation process lead to a yield decrease, but also the pulverization into the particulates results in discontinuity of the process, bring forth ineffectiveness in terms of time and costs.

Korean Patent No. 0695834 disclosed a pharmaceutical composition with an enhanced stability by preparing a mixture of an antioxidant and a sirolimus derivative that is sensitive to an oxidation reaction. This patent explicitly stated that the stability can be improved by preparing a mixed precipitates of a sirolimus derivative and an antioxidant. However, such process can be carried out only after complicated procedures including the steps of synthesizing a sirolimus derivative, dissolving the synthesized sirolimus derivative, adding an antioxidant thereto and subjecting the resulting mixture to an initial stirring, adding water dropwise thereto to provide a suspension, washing the suspension with water and an organic solvent, and then drying the obtained product under vacuum. Moreover, BHT is a phenol based antioxidant, raising a lot of controversy over hyperactivity disorder for some children and carcinogenicity, and thus some food manufacturers have voluntarily limited its use for an additive.

Korean Patent No. 0541198 disclosed a pharmaceutical composition in the form of a micro-emulsion pre-concentrate comprising a sirolimus derivative and a water-soluble carrier consisting of: 1) a hydrophilic phase containing dimethylisosorbide, 2) a lipophilic phase, and 3) a surfactant. This patent disclosed a formation of uniform micro-emulsion by bringing the micro-emulsion pre-concentrate formulation into contact with water. However, such composition has a very high viscosity causing an inconvenience in the preparation process and its examples also show that more than 14% of the main component was observed to be decomposed after a short-term (4 week) storage at 25° C., indicating that such composition has a very low level of stability.

### SUMMARY OF THE INVENTION

Thus, the object of the present invention is to develop a technique capable of remarkably improving solubility and stability of a sirolimus derivative in a very simple manner, without using any antioxidant and/or stabilizer harmful to living bodies.

Accordingly, an embodiment provides a process of preparing a solubilized and stabilized formulation of a sirolimus derivative, which comprises the steps of dissolving a sirolimus derivative in a solvent, and contacting the solution of the sirolimus derivative with a water-soluble carrier, to disperse the sirolimus derivative in the water-soluble carrier.

Other embodiment provides a formulation of a sirolimus derivative with improved solubility and stability, which is prepared by the preparation process as described above.

### BRIEF DESCRIPTION OF THE DRAWINGS

FIGS. 1A to 1D are scanning electronic microscope (SEM) images of non-treated HPMC (FIG. 1A), Example 2 (FIG. 1B), Example 4 (FIG. 1C) and Comparative Example 1 (FIG. 1D), respectively. (with magnification power of 200)

FIG. 2 shows graphs illustrating the elution properties of everolimus in distilled water for Examples 1 to 6 and Comparative Examples 1 and 2.

### DETAILED DESCRIPTION OF THE EMBODIMENT

The present inventors developed a technique, wherein a sirolimus derivative solution and a water-soluble carrier medium are brought into contact with each other through a simple process such as a wet granulation or a spray drying by using a high speed shearing mixer, a fluid bed granulator, or any corresponding equipment, thereby allowing the sirolimus derivative to have not only significantly improved water solubility but also a higher level of stability without using any antioxidant/stabilizer, especially a synthetic antioxidant including butylated hydroxytolune (BHT) described in Korean Patent No. 0695834, which was found to be detrimental to a human body, to complete the present invention.

An embodiment of the present invention provides a process for preparing a solubilized and stabilized formulation of a sirolimus derivative, which comprises the steps of dissolving a sirolimus derivative in a solvent, and contacting the solution of the sirolimus derivative with a water-soluble carrier, to disperse the sirolimus derivative in the water-soluble carrier.

Other embodiment provides a formulation of a sirolimus derivative with an improved solubility and stability, prepared by the foregoing preparation process.

Hereinafter, the present invention will be explained in more detail.

The effective ingredient of the present invention is a sirolimus derivative or a pharmaceutically acceptable salt thereof.

The term “sirolimus derivatives” as used herein inclusively refer to sirolimus, its structurally similar homologous, and its derivatives.

For example, the sirolimus derivative can be one or more selected from the group consisting of sirolimus; 16-O-substituted sirolimus derivatives (for example, see WO94/02136); 40-O-substituted sirolimus derivatives, for example, O-aryl or O-alkyl rapamycins (see U.S. Pat. No. 5,258,389 and WO94/09010); carboxylic acid substituted ester derivatives (see WO92/05179), amide ester substituted sirolimus derivatives (see U.S. Pat. No. 5,118,677), carbamate substituted sirolimus derivatives (see U.S. Pat. No. 5,118,678), fluorinated ester substituted sirolimus derivatives (see U.S. Pat. No. 5,100,883), acetal substituted sirolimus derivatives (see U.S. Pat. No. 5,151,413), silyl ether substituted sirolimus derivatives (see U.S. Pat. No. 5,120,842), methylene substituted sirolimus derivatives (see WO93/11130; methylene rapamycin), methoxy substituted sirolimus derivative (see WO94/02136), hydroxyethyl substituted sirolimus derivatives (see Korean Patent No. 0308598), alkenyl substituted sirolimus derivatives (see WO94/02385 and WO95/14023); 32-O-dihydro or 32-O-substituted sirolimus derivatives (see U.S. Pat. No. 5,256,790); and 32-deoxorapamycin and 16-pent-2-ynyloxy-32(S)-dihydrorapamycin (see PCT/EP96/02441). All the documents as described above are incorporated herein by reference.

Specifically, the sirolimus derivatives can be, but are not limited to, at least one selected from the group consisting of sirolimus, everolimus (40-O-(2-hydroxy)ethyl-rapamycin), and the like. The present invention can be applied to any sirolimus derivative. The pharmaceutically acceptable salts of the sirolimus derivative comprise any acidic or basic salts and their stereochemical isomers. The salts comprise any one that can maintain an activity of their parent compounds and does not lead to any undesirable effect. They are not particularly limited, comprising all of organic salts and inorganic salts. As examples of the acidic salt, mentions may be made of a salt of acetic acid, nitric acid, aspartic acid, sulfonic acid, sulfuric acid, maleic acid, glutamic acid, formic acid, succinic acid, phosphoric acid, phthalic acid, tannic acid, tartaric acid, hydrobromic acid, propionic acid, benzene sulfonic acid, benzoic acid, stearic acid, butyric acid, bicarbonic acid, bisulfuric acid, bitartaric acid, oxalic acid, butylic acid, calcium edetate, carbonic acid, chlorobenzoic acid, citric acid, edetic acid, toluene sulfonic acid, edisylic acid, fumaric acid, gluceptic acid, pamoic acid, gluconic acid, glycollylarsanilic acid, methyl nitric acid, polygalacturonic acid, hexylresorcinoic acid, malonic acid, hydrabamic acid, hydrochloric acid, hydroiodic acid, hydroxynaphtholic acid, isethionic acid, lactobionic acid, mandelic acid, estorlinic acid, mucic acid, napsilic acid, muconic acid, p-nitromethansulfonic acid, hexamic acid, pantothenic acid, monohydrogen phosphoric acid, dihydrogen phosphoric acid, salicylic acid, sulfamic acid, sulfanilic acid, methansulfonic acid, or teoclic acid. Moreover, the types of the basic salts include, for example, an ammonium salt, an alkali metal or alkaline earth metal salt such as lithium, sodium, potassium, magnesium, and calcium salts, for example, a salt having an organic base such as benzathine, N-methyl-D-glucamine, and hydrabamine salts, and for example, a salt having an amino acid such as arginine and lysine. In addition, such salts can be transformed into a free acid or a free base by treating them with an appropriate acid or base. An “addition salt” includes a solvate which can be formed by the sirolimus derivatives and a salt thereof. The solvate compound can be, for example, a hydrate or an alcoholate.

The solubilized and stabilized formulations of the sirolimus derivative of the present invention can further comprise other effective ingredients capable of being combined with the sirolimus derivatives and their salts.

Types of the solubilized and stabilized formulations of the sirolimus derivative are characterized in that the sirolimus derivative is uniformly dispersed in the water-soluble carrier. Depending on the preparation process, the sirolimus derivatives can be distributed in the water-soluble carrier in the form of a carrier surface attachment or a homogeneous distribution of the carrier and the drugs. The types of the formulations of the present invention can be referred to as a wet granule or a non-coprecipitated solid dispersion in a typical and broad meaning by a person of ordinary skill in the art. The formulation of the present invention is prepared as uniform granules with minimizing a change in the carrier properties, thereby presenting groundbreaking advantages that additional processes such as pulverization of the particulates can be omitted, in comparison with conventional techniques for the sirolimus formulations (for example, see Korean Patent No. 0352943).

The present invention is characterized in that the dispersion the sirolimus derivative in the water-soluble carrier is achieved through a wet process using a solution wherein the sirolimus derivative is dissolved in a solvent.

In order to carry out the wet process, the preparation process of the present invention is characterized by comprising a step of dissolving a sirolimus derivative in water, an organic solvent, or a mixed solvent of water and an organic solvent.

The organic solvent may be a single solvent or a mixture of solvents, and it may be any polar or non-polar organic solvent capable of dissolving or suspending the sirolimus derivative and thereby allowing it to be sprayed. Solvents suitable for use in the preparation of the solid-phase dispersion formulation of the present invention may be a highly volatile solvent with an excellent solubility for the sirolimus derivative, and may be one or more selected from the group consisting of a linear or branched alcohol having 1 to 10, preferably 1 to 5 carbon atoms, such as methanol, ethanol, or isopropanol; an ester having 3 to 10, preferably 3 to 6 carbon atoms, such as ethyl acetate; a polar or non-polar ester having 3 to 10, preferably 3 to 6 carbon atoms, such as diethyl ether; a polar or non-polar ketone having 1 to 10, preferably 1 to 5, such as acetone; and a halogenated hydrocarbon having 1 to 10, preferably 1 to 5, such as dichloroethane. Among them, the most convenient solvent is ethanol, considering the properties such as the solubility for the sirolimus derivative.

The amount of the solvent as used may be adjusted within such a range that the sirolimus derivative is sufficiently dissolved and its granulation is facilitated on contact with the water-soluble carrier. For example, the solvent may be used in an amount of 0.01 to 1000 mL, preferably 0.05 to 500 mL, with respect to 1 g of the sirolimus derivative, but may be not limited thereto. The amount of the solvent as used may be properly adjusted by a person of ordinary skill in the art depending on the types of the sirolimus derivative and the solvent. When the amount of the solvent is less than the above range, it is difficult to achieve uniform contact between the sirolimus derivative and the water-soluble carrier, while the excess of the above range leads to time for the process and a change in the properties of the carrier. Therefore, it is advantageous that the amount of the solvent as used be in the foregoing range.

Further, the present invention is characterized in that the step of contacting the solution of the sirolimus derivative in the solvent with the water-soluble carrier to disperse the sirolimus derivative in the water-soluble carrier can be carried out through
1) mixing the sirolimus derivative solution with the water-soluble carrier, for example, by using a high speed shearing mixer, and drying the same; or
2) spraying the sirolimus derivative solution onto the fluidizing water-soluble carrier, for example, by using a fluid bed granulator, and drying the same.

As described above, the step of contacting the solution of the sirolimus derivative in the solvent with the water-soluble carrier to disperse the sirolimus derivative in the water-soluble carrier may be carried out by using a high speed shearing mixer, a fluid bed granulator, or any wet granulation equipment corresponding thereto. As the wet granulation equipment, one can use various vessel rotated-, vessel fixed-, or fluidized mixers including Hobart mixer and CF granulator. If necessary, one can use an oscillator granulator, a motorized granulator, a screw extruding granulator, a centrifugal granulator, a crushing granulator, or the like. With using such equipment, the sirolimus derivative can be dispersed in the water-soluble carrier while being granulated so that the step of bring the sirolimus derivative solution into contact with the water-soluble carrier to disperse the sirolimus derivative in the water-soluble carrier can comprise a granulation process.

When the sirolimus derivative solution is brought into contact with the water-soluble carrier, the ratio between their amounts (i.e., the weight of the sirolimus derivative solution:the weight of the water-soluble carrier) may range from 1:0.01 to 1:1000, preferably from 1:0.05 to 1:500, for example, from 1:0.1 to 1:300, from 1:0.5 to 1:100, from 1:1 to 1:50, or from 1:5 to 1:30. The proportion of the sirolimus solution below the above range can bring about difficulties in carrying out a uniform spraying or dispersion, while the proportion of the sirolimus solution exceeding the above range can cause the processing time to be unnecessarily extended. Therefore, it is advantageous that the ratio between the sirolimus derivative solution and the water-soluble carrier be within the above range.

According to an embodiment of the present invention, a stabilized formulation of the sirolimus derivative can be prepared by using a fluid bed granulator or a high speed shearing mixer. In other words, the embodiment is directed to a process wherein the water-soluble carrier is accurately weighed and put into a fluid bed granulator or a high speed shearing mixer for a pharmaceutical use and the sirolimus derivative solution or an organic solvent comprising the sirolimus derivative is sprayed thereto and dried to give an oral formulation.

In the high speed shearing mixer process, the mixer may be operated at a speed of 1 to 1000 rpm, preferably from 5 to 500 rpm, or in case of a chopper, at a speed from 1 to 10000 rpm, preferably from 5 to 5000 rpm. At the speed above or below the foregoing range, a uniform mixing of the sirolimus derivative may not ensured such that the process using the high speed mixer is preferably carried out under the above condition. However, a person of ordinary skill in the art can properly adjust the above condition depending on the types and the size of the high speed shearing mixer and the present invention is not limited thereto.

In the fluid bed granulator process, the granulator may be operated under the conditions of a spraying pressure of 0.01 bar or higher, preferably 0.1 bar or higher, for example 0.01 to 10 bar, or 0.1 to 10 bar and a processing temperature of 0 to 120° C., preferably 0 to 60° C. The spraying pressure below the above range can cause difficulties in ensuring uniform spraying of the sirolimus solution and the process carried out at a processing temperature over 60° C. can lead to the degradation of the drug. Therefore, the process of the fluid bed granulator is preferably operated under the above-mentioned conditions. However, a person of ordinary skill in the art can properly adjust these conditions depending on the types and the size of the equipment and the present invention is not limited thereto.

The preparation process of the present invention can further comprise a step of filtering and/or drying the product from the step of bring the sirolimus derivative solution into contact with the water-soluble carrier to obtain a final granulated formulation. For example, one can carry out an additional step wherein a mixed raw material for granulation is sieved by using a sieve of 10 to 50 mesh and then dried (for example, through a high-speed drying or a tray drying) until the loss on drying is less than 10% (w/w).

The sirolimus derivative granules as obtained by the preparation process of the present invention are characterized in that the average diameter of the particles is uniformly maintained to correspond to the granularity of the water-soluble carrier. The average particle size is dependent on the types of the water-soluble carrier, being in the order of about 0.01 to 500 um, but the present invention is not limited thereto.

The preparation process of the present invention is characterized in that the product from such granulation step (optionally including subsequently sieving and drying) can be mixed with other pharmaceutical excipients and directly tableted without any additional process such as a pulverization (grinding) process (e.g., milling). If necessary, the stabilized formulation as obtained can be mixed with other pharmaceutical excipients and tableted in a continuous manner for the process.

The content of the sirolimus derivative in the formulation can be about 0.01 to about 40% by weight, for example, 3 to 20% by weight based on the total weight of the formulation. The content of the water-soluble carrier in the formulation can be 5 to 99.99% by weight, for example, 10 to 95% by weight based on the total weight of the formulation. The proportion of the water-soluble carrier exceeding the above range can cause problems such as an increase in a tablet dosage or a delay of disintegration or elution of the tablet, while the proportion of the water-soluble carrier below the above range can lead to a significant decrease in the effect of solubilization and stabilization. Therefore, it is advantageous that the proportion of the water-soluble carrier be within the above range.

The water-soluble carrier comprises a water-soluble polymer, specifically a cellulose derivative selected from the group consisting of hydroxy propyl methylcellulose (HPMC), hydroxy propyl methylcellulose phthalate, and poly vinyl pyrrolidone (PNP). HPMC with a low apparent dynamic viscosity, e.g. below 100 cps (for example, 0.1 cps or higher but less than 100 cps) as measured at 20° C. for a 2% by weight aqueous solution, e.g., below 50 cps (for example, 0.1 cps or higher but less than 50 cps), preferably below 20 cps (for example, 0.1 cps or higher but less than 20 cps), for example HPMC of 3 cps can be used, but the present invention is not limited thereto.

HPMC, including HPMC of 3 cps, is commercially available under the trade name Pharmacoat 603 from the Shinetsu Co. PVP is available, for example, under the name Povidone (Handbook of Pharmaceutical Excipients), and a PNP having an average molecular weight between about 8,000 and about 50,000 Daltons is preferred.

Besides, the water-soluble carrier can be at least one selected from the group consisting of:
hydroxy propyl cellulose (HPC) or a derivative thereof;
polyethylene glycols (PEG) such as PEGs having an average molecular weight between 1000 and 9000 Daltons, e.g. between about 1800 and 7000, including PEG 2000, PEG 4000 or PEG 6000 (Handbook of Pharmaceutical Excipients);
saturated polyglycolised glycerides, (for example having an average molecular weight of 1000 to 15000 g/mol) including Gelucir™ (e.g. Gelucir 44/14, 53/10, 50/13, 42/12, or 35/10); and
cyclodextrins, for example a β-cyclodextrin or an α-cyclodextrin (e.g., β-cyclodextrins; methyl-β-cyclodextrin; dimethyl-β-cyclodextrin, hydroxypropyl-β-cyclodextrin; glycosyl-β-cyclodextrin; maltosyl-β-cyclodextrin; sulfo-β-cyclodextrin; sulfo-alkyl ethers of β-cyclodextrin such as sulfo-C₁₋₄-alkyl ethers; α-cyclodextrins; glucosyl-α-cyclodextrin, maltosyl-α-cyclodextrin, and the like).

In another embodiment, the water-soluble carrier can be at least one selected from the group consisting of hypromellose, polyvinyl alcohol, polyethylene oxide, polyethylene glycol, polyvinyl pyrrolidone, hydroxypropyl cellulose, hydroxy ethyl cellulose, vinyl pyrrolidone-vinyl acetate copolymer arginate, calcium carboxymethyl cellulose, sodium carboxymethyl cellulose, carbomer, carrageenan, chitosan, guar gum, and dimethyl aminoethyl methacrylate-buthylmethacrylate-methyl methacrylate copolymer (Eudragit E).

The carrier medium may further comprise at least one pharmaceutically acceptable carrier or filler selected from the group consisting of a water-soluble or water-insoluble saccharose, lactose, and microcrystalline cellulose.

In addition, the water-soluble carrier may further comprise one or more surfactants, for example at least one selected from the group consisting of a non-ionic, ionic, anionic, and amphoteric surfactants. As examples of suitable surfactants, mentions may be made of at least one selected from the group consisting of the following compounds:
polyoxyethylene-polyoxypropylene co-polymers or block co-polymers (for example, having an average molecular weight of 1000 to 15000 g/mol) known, for example, under the trade names Pluronic or Poloxamer, preferably, polyoxyethylene-polyoxypropylene block polymer (e.g., Poloxamer 188 commercially available from the BASF company);
ethoxylated cholesterins known, for example, under the trade name Solulan commercially available from the Amerchol company (e.g., Solulan C24);
vitamin derivatives, e.g. vitamin E derivatives such as tocopherol polyethylene glycol succinate (TPGS) available from the Eastman company;
sodium dodecylsulfate or sodium laurylsulfate;
a bile acid or salt thereof, for example cholic acid, glycolic acid or a salt, e.g. sodium cholate; and
lecithin.

If present in the formulation of this invention, the surfactant(s) is generally in an amount of up to about 20%, for example 1 to 15% by weight.

One or more disintegrating agent may be included in the compositions of this invention. As the disintegrating agent, one may use any pharmaceutically acceptable, typical disintegrating component. For example, it can be at least one selected from the group consisting of vinyl pyrrolidone (Polyplasdone available commercially from the ISP company; Handbook of Pharmaceutical Excipients); sodium starch glycolate (e.g., sodium starch glycolate commercially available from the Generichem company); and crosscarmelose sodium (e.g., available under the trade name Ac-di-sol from FMC Corporation).

If present in the formulations of this invention, the disintegrating agent(s) can be in an amount of 0.01 to 50% by weight, preferably 0.1 to 30% by weight.

If necessary, an antioxidant and/or a stabilizer may be further included in the formulations of this invention in an amount of up to about 1% by weight, for example between 0.05 and 0.5% by weight. Examples of the antioxidant and/or the stabilizer include butylated hydroxytoluene, DL-α-tocopherol, propyl gallate, ascobyl palmitate, malonic acid, fumaric acid, and the like.

In the formulations of the present invention, such antioxidants and/or stabilizers are only an optional ingredient, without which the formulations of the present invention show sufficient stability by itself. For example, as confirmed by Test Example 3, the formulations of the present invention without including any antioxidant and/or stabilizer have a drug (i.e., the sirolimus derivative) content of 80% or higher by weight, preferably no less than 82% by weight, for example 80 to 95% by weight, or 82 to 90% by weight with respect to the initial content, after a 60 hour storage at a temperature of 80° C.

If the addition of a surfactant, a disintegrating agent, and, if necessary, an antioxidant and/or a stabilizer is made in the preparation process of the present invention, they can be applied in the wet process step or in the step of mixing the obtained granules with other excipients. Preferably, the surfactant or the antioxidant and/or the stabilizer can be added in the wet process step while the disintegrating agent can be added in the mixing step after the wet process, but the present invention is not limited thereto.

The formulations of the present invention can be formulated as a composition for oral administration. Such oral compositions of the sirolimus derivative are useful for the known indications of the sirolimus derivative, e.g. the following conditions:
a) treatment and prevention of organ or tissue allo- or xeno-transplant rejection, e.g. for the treatment of recipients of e.g. heart, lung, combined heart-lung, liver, kidney, pancreatic, skin or corneal transplants, and for the prevention of graft-versus-host disease, such as following bone marrow transplantation.
b) treatment and prevention of autoimmune disease and of inflammatory conditions, in particular inflammatory conditions with an etiology including an autoimmune component such as arthritis (for example, rheumatoid arthritis, arthritis chronica progrediente and arthritis deformans) and rheumatic diseases.
c) treatment and prevention of asthma.
d) treatment of multi-drug resistance (MDR). MDR is particularly problematic in cancer patients and AIDS patients who will not respond to conventional chemotherapy because the medication is pumped out of the cells by Pgp. The present formulations are therefore useful for enhancing the efficacy of other chemotherapeutic agents in the treatment and control of multidrug resistant conditions such as multidrug resistant cancer or multidrug resistant AIDS.
e) treatment of proliferative disorders, e.g. tumors, hyperproliferative skin disorder and the like.
f) treatment of fungal infections.
g) treatment and prevention of inflammation, especially in potentiating the action of steroids.
h) treatment of infection, especially infection by pathogens having Mip or Mip-like factors.
i) treatment of overdoses of FK-506 and other macrophilin binding immune-suppressants.

The present invention provide a pharmaceutical composition which is capable of being administered of its own only for the effective amount of the oral administration into a human body, but includes at least one excipient. The pharmaceutical composition of the present invention can be in the form of a tablet, a capsule, a troche, dispersions, powders, solutions, or granules, all of which can be prepared by typical pharmacological methods (for example, the method as exemplified in ‘Remington's Pharmaceutical Science, Mack Publishing Co.’). The pharmacological composition provided by the present invention can include different excipients according to corresponding objectives, the types of which are fully described in ‘Handbook of Pharmaceutical Excipients, 6th edition, Pharmaceutical Press.’

As described above, the present invention is directed to a process for preparing a formulation of a sirolimus derivative wherein the water solubility and the stability of the sirolimus derivative commercially available for an mTOR inhibitor are remarkably enhanced through a simple process. According to the present invention, the formulation of the hydrophilic carrier and the sirolimus derivative can be prepared by using a high speed shearing mixer or a fluid bed granulator and in a simple process of a wet process or a spray drying corresponding thereto, and thereby the sirolimus derivative highly sensitive to an oxidation reaction can be surprisingly stabilized.

Accordingly, without further adding an antioxidant or a stabilizer, the preparation process of the present invention can prepare the sirolimus derivative formulations with a higher level of stability, and the formulations of the present invention thus obtained has such an enhanced water solubility for the drug that no additional process is required to improve an elution rate. Moreover, it can be prepared by a simple process and thus one can expect a high production efficiency.

The present invention provides the following effects:

First, through a simple process, one can enhance the water solubility and the stability of the sirolimus derivative, and also can prepare the sirolimus derivative formulations with excellent properties.

Secondly, without adding any synthetic antioxidant or other stabilizer, the sirolimus derivative can be more stabilized.

Thirdly, it is possible to enhance an elution rate of the sirolimus derivative and to increase its bioavailability.

Forthly, it is possible to make formulations directly in the step of preparing raw materials without a solidification process, thereby providing a stable composition and thus facilitating the storage and distribution thereof.

### EXAMPLE

Hereinafter, for better understanding of the present invention, it will be explained in detail with reference to the examples. However, the examples of the present invention can be modified in different manners and the scope of the present invention should not be construed to be limited by those examples. The examples of the present invention are presented in order to explain the present invention more perfectly in view of a person of ordinary skill in the art.

### Example 1

### Small Scale Wet Granulation Process

0.3 g of everolimus was added to 6 mL of absolute ethanol and dissolved by stirring the same with a magnetic stirrer at 100 rpm for 5 minutes. 6 mL of the obtained ethanol solution of everolimus was added dropwise to 4.5 g of HPMC (having a viscosity of 3 cps as measured at 20° C. for a 2 wt % aqueous solution) and mixed by using a mortar and a pestle for 5 minutes. After being uniformly spread out, the granules thus obtained were dried in a dry oven at 30° C. for 1 hour to give 4.8 g of everolimus granules.

### Examples 2 and 3

### High Speed Shearing Mixer (HSM) Process

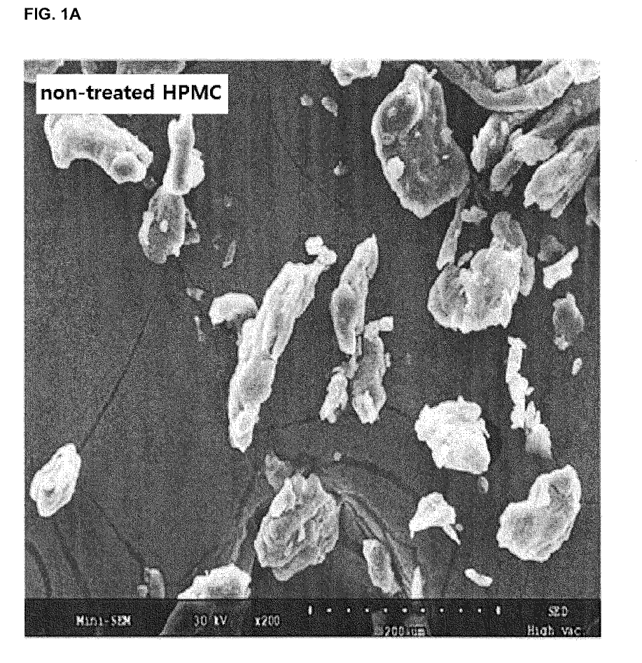

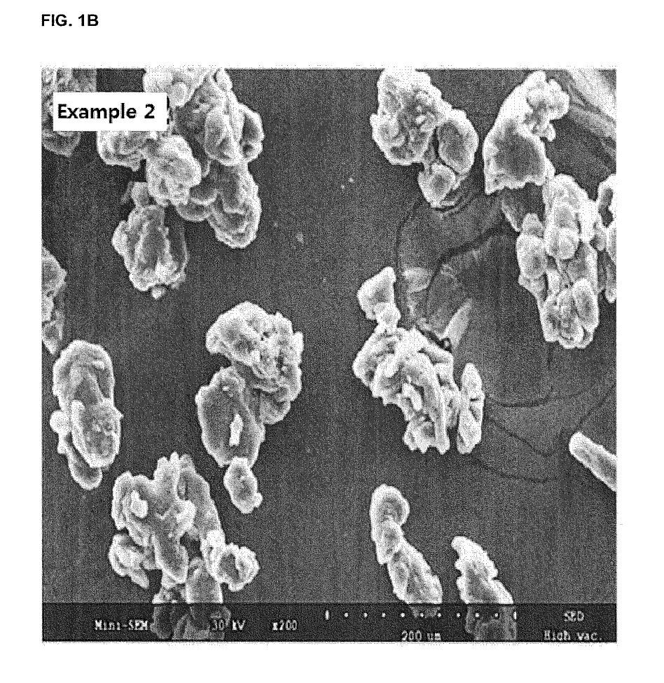

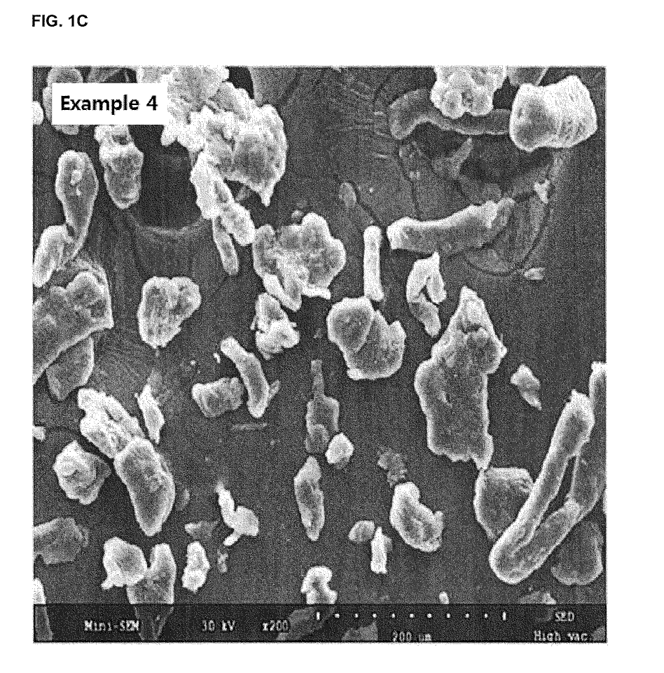

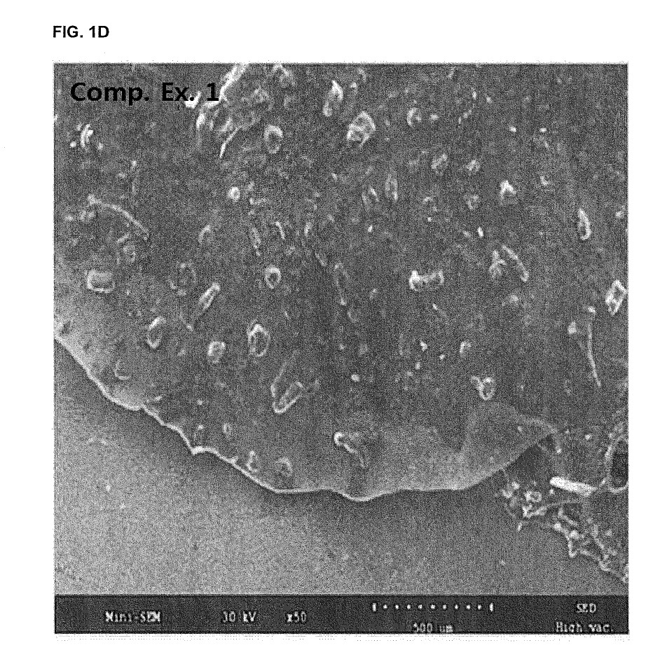

A high speed shearing mixer (Diosna Co.) was used for mixing 150 g of HPMC (having a viscosity of 3 cps as measured at 20° C. for a 2 wt % aqueous solution) under the conditions of 300 rpm for a mixer and 500 rpm for a chopper. A solution comprising 10 g of everolimus dissolved in 100 mL of absolute ethanol was slowly added dropwise thereto and then mixed for about 5 minutes. For Example 2, the resulting mixture was tray-dried at 30° C. for 1 hour, and for Example 3, it was dried with a high speed drying machine (Retsch Co., model name: TG200) at 40° C., and in both examples, 160 g of everolimus granules were obtained (see FIG. 1B). The loss weight during the process was within 4 wt % with respect to its theoretical weight.

### Example 4

### Fluid Bed Granulator (FBG) Process

After 20 g of everolimus was dissolved in 400 mL of absolute ethanol, about 400 mL of the everolimus solution thus obtained was sprayed at a pressure of 0.5-2 bar onto 300 g of HPMC (with a viscosity of 3 cps as measured at 20° C. for a 2 wt % aqueous solution) as fluidized under a proper airflow pressure by using a fluid bed granulator (mini Glatt, Glatt Co.), and then the resulting product was dried at a temperature of 20-40° C. to provide 320 g of everolimus granules (see FIG. 1C). The loss weight during the process was within 5 wt % with respect to its theoretical weight.

### Example 5

### Combination of a Raw Material Synthesis and a High Speed Shearing Mixer Process

Everolimus was synthesized from sirolimus according to the method as set forth in Korean Patent No. 0308598 (see Example 8) and a high purity everolimus solution was obtained by using a Prep LC. The Prep LC as used was YoungJin-DAC model. DAISO ODS GEL was used as a filler, a solution of 75% (v/v) methanol: 25% (v/v) first distilled water was used as a mobile phase, and acetone and isopropyl alcohol was used as a filling solvent. The solution obtained from the LC, corresponding to 10 g of everolimus, was directly diluted 10 times by volume with ethanol without any additional drying process.

150 g of HPMC (with a viscosity of 3 cps as measured at 20° C. for a 2 wt % aqueous solution) was mixed by using a high speed shearing mixer at room temperature at 300 rpm for a mixer and 500 rpm for a chopper, and at the same time, the diluted solution of everolimus was added dropwise thereto to provide everolimus granules. The resulting product was dried at 40° C. by using a high speed drier (Retsch Co., model name: TG200) to give 160 g of everolimus granules. The granules as prepared had a similar granularity to that of HPMC as used, and the loss weight during the process was within 5 wt % with respect to the theoretical weight.

### Example 6

### Combination of a Raw Material Synthesis and a Fluid Bed Granulator Process

Everolimus was synthesized from sirolimus according to the method as set forth in Korean Patent No. 0308598 (see Example 8) and a high purity everolimus solution was obtained by using a Prep LC. The Prep LC as used was YoungJin-DAC model. DAISO ODS GEL was used as a filler, a solution of 75% (v/v) methanol: 25% (v/v) first distilled water was used as a mobile phase, and acetone and isopropyl alcohol was used as a filling solvent. The solution obtained from the LC, corresponding to 20 g of everolimus, was directly diluted 10 times by volume with ethanol without any additional drying process. After that, while the diluted solution was sprayed at a pressure of 0.5-2 bar onto 300 g of HPMC (with a viscosity of 3 cps as measured at 20° C. for a 2 wt % aqueous solution) fluidized under a proper airflow pressure by using a fluid bed granulator (mini Glatt, Glatt Co.), the resulting product was dried at a temperature of 20-40° C. to prepare 320 g of everolimus granules (see FIG. 1C). The loss weight during the process was within 8 wt % with respect to the theoretical weight.

### Comparative Example 1

### Preparation of a Co-Precipitated Solid Dispersion

According to the method as disclosed in Example 1 of Korean Patent No. 0352943, a co-precipitated composition comprising everolimus, HPMC (with a viscosity of 3 cps as measured at 20° C. for a 2 wt % aqueous solution), and lactose (200 mesh) with a ratio of 1:9:1 (based on the weight) was prepared. Specifically, 10 g of everolimus, 90 g of HPMC, and 10 g of lactose were added to a 1:1 mixed solution of ethanol and acetone (based on the weight) and stirred for 2 hours. The resulting product was subjected to a tray-drying at 30° C. to produce a formulation. (see FIG. 1D)

### Comparative Examples 2 and 3

For comparison with the present invention, in Comparative Example 2, everolimus (40-O hydroxyethyl rapamycin) was utilized. In Comparative Example 3, a formulation was obtained by adding 0.2% (w/w) of an antioxidant, butylated hydroxy toluene (BHT) in the same manner as set forth in Korean Patent No. 0695834 (see Example 2)

### Test Example 1

### Evaluation for Characteristics of the Formulation and Easiness of the Process

For the compositions as prepared by the above preparation process, their surfaces were observed and compared with each other by using scanning electron microscope (SEM) images. Each of the formulations as prepared in accordance with the above mentioned examples was subjected to a Pt/Pd coating with a thickness of about 15 nm and was subjected to a SEM analysis with the scanning electron microscope (Hitachi S-4300). Furthermore, for comparison of the characteristics of each composition, a SEM image for a non-treated HPMC was taken together.

FIGS. 1A to 1D are SEM images of the non-treated HPMC, and the formulations as prepared in Examples 2 and 4, and Comparative Example 1, respectively. FIGS. 1A to 1D show that granules of Example 2 (FIG. 1B) and Example 4 (FIG. 1C) as prepared according to the present invention have uniform characteristics with no significant difference from those of the non-treated HPMC (FIG. 1A). By contrast, in Comparative Example 3 (FIG. 1D), despite the same drying process as Example 2, HPMC was dissolved in the organic solvent and a part of HPMC was swelled and then dried to form a film-like composition.

For the granules of Examples 2 and 4, which had not been subjected to a milling process, the angle of repose was measured to be 40.5° and 40.8°, respectively, indicating their excellent fluidity. In contrast, it was impossible to measure the angle of repose for Comparative Example 1 because of the formation of the film-like composition of HPMC and the drug. The composition of Comparative Example 1 did not exhibit a fluidity corresponding to those of Examples 2 and 4 until its average grain size was controlled to be 200-300 μm through an additional milling process by using a jet milling. These results show that a milling process is a requisite process in order for the composition of Comparative Example 1 to be mixed with a pharmaceutical excipient and prepared as a tablet.

In case of the composition of Comparative Example 1, the steps of spray-drying it, subjecting it to a milling machine, collecting it after the milling lead to an increased processing time by 30% or more in comparison with those of Examples 2 and 4. Furthermore, the obtained amounts of Examples 2 and 4 were 95% or more of the initial amount, while the obtained amount of Comparative Example 1 decreased to 60% with respect to the initial amount, and this result indicates the inefficiency of Comparative Example 1.

From the above test results, it can be found that preparation of the sirolimus derivative granules in accordance with the present invention makes it possible to produce a desired type of granules simply with a wet granulation process, and to prepare a granulated formulation with uniform characteristics even with no need to carry out an additional process such as a milling.

### Test Example 2

### Evaluation for an Elution Property

The granules of the examples were mixed with pharmaceutical excipients as set forth in Table 1 and then tableted to give a tablet. A direct compressing method was used to prepare a tablet comprising 10 mg of a sirolimus derivative with a hardness of 13 to 15 Kp. However, the formulation prepared from Comparative Example 1 had difficulties in being mixed with the pharmaceutical excipients of Table 1 and also met with an obstacle in a tableting process so that it was subjected to an additional milling process and controlled to have a granularity of 200 to 300 μm before being mixed with the excipients and tableted.

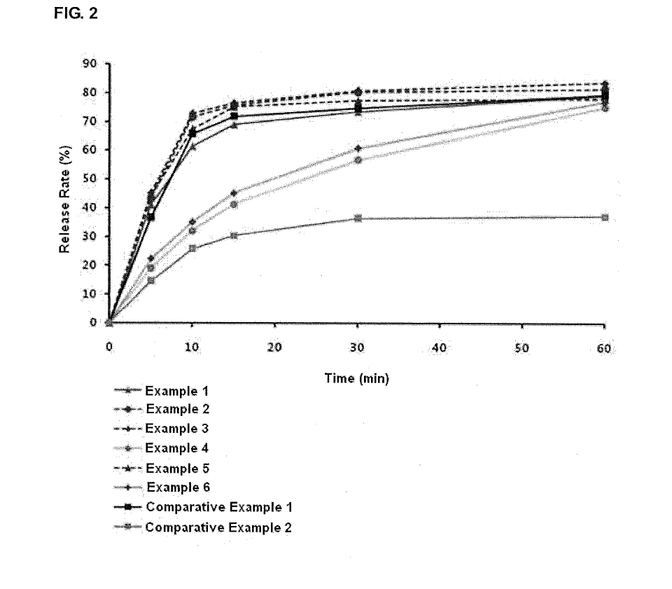

The tablets as obtained were evaluated for their elution property. 900 mL of an elution liquid (distilled water) was put into an elution tester and the granules as obtained in Examples 1 to 6 were taken in an amount corresponding to a dosage of 10 mg of everolimus and subjected to an elution test at 37.5° C. with 50 cycles per a minute by using a paddle method for 2 hours. For each clinical specimen taken in accordance with a predetermined schedule, the concentration of everolimus in the elution liquid was quantified by using a high performance liquid chromatography. The results are shown in FIG. 2.

In comparison with the present invention, the co-precipitated solid dispersion (Comparative Example 1) and the non-treated everolimus (Comparative Example 2) were also tableted in the same manner as above and subjected to an elution test. The results are also shown in FIG. 2.

As shown in FIG. 2, the non-treated everolimus (Comparative Example 2, purchased from BIOCON Co.), which was water-insoluble, showed an elution rate of about 37%, while all the formulations of Examples 1 to 6 prepared conveniently from the wet-granulation process exhibited an elution rate of about 80% after 1 hour, which was a level comparable to the composition of Comparative Example 1 obtained from the co-precipitation process.

TABLE 1
Pharmaceutical Composition

| Raw materials | mg per tablet |
|---|---|
| Example 1 | the amount |
| Example 2 | corresponding |
| Example 3 | to 10 mg of |
| Example 4 | everolimus |
| Example 5 | |
| Example 6 | |
| Comparative Example 1 | |
| Comparative Example 2 | |
| Anhydrous Lactose | 188.75 |
| Crospovidone | 100 |
| Microcrystalline cellulose | 50 |
| Magnesium stearate | 1.25 |

### Test Example 3

### Evaluation for Stability

For Examples 1 to 6 and Comparative Examples 2 and 3, their stability was evaluated and the results were compared. Each of the samples was placed in a glass vial and stored at 80° C. for 60 hours, and then the content of the drug was quantified by using a high performance liquid chromatography. The results are shown in Table 2.

TABLE 2
Results of comparison of the stability at 80° C. for Examples 1 to
6 and Comparative Examples 2 and 3

| | | | | | | | Comparative | Comparative |
|---|---|---|---|---|---|---|---|---|
| Content | Example 1 | Example 2 | Example 3 | Example 4 | Example 5 | Example 6 | Example 2 | Example 3 |
| initial (wt %) | 100.4 | 98.6 | 98.3 | 99.4 | 103.0 | 98.7 | 100.4 | 99.1 |
| After 60 hours | 84.6 | 87.2 | 86.2 | 86.5 | 86.3 | 84.0 | 23.5 | 57.3 |
| (wt %) | | | | | | | | |

After being left for 60 hours, the granules prepared from Examples 1 to 6 have a content of 84-87%, showing the stability 3.5 to 3.7 times higher than that of Comparative Example 2 (23.5%). Also, they showed a surprisingly increased stability by 1.5 times or more with respect to that of Comparative Example 3 (57.3%).

What is claimed is:

1. A process of preparing a formulation of a sirolimus derivative, comprising the steps of:
mixing a sirolimus derivative with water, an organic solvent, or a mixed solvent thereof; and
contacting the obtained solution of the sirolimus derivative with a water-soluble carrier, to disperse the sirolimus derivative in the water-soluble carrier.

2. The process according to claim 1, wherein the step of contacting the obtained solution of the sirolimus derivative with the water-soluble carrier, to disperse the sirolimus derivative in the water-soluble carrier, is carried out by using a high speed shearing mixer or a fluid bed granulator.

3. The process according to claim 1, wherein the sirolimus derivative is one or more selected from the group consisting of sirolimus, 16-O-substituted sirolimus derivatives, 40-O-substituted sirolimus derivatives, ester derivatives of a carboxylic acid substituted sirolimus, carbamate substituted sirolimus derivatives, fluorinated ester substituted sirolimus derivatives, acetal substituted sirolimus derivatives, silyl ether substituted sirolimus derivatives, methylene substituted sirolimus derivatives, methoxy substituted sirolimus derivative, hydroxyethyl substituted sirolimus derivatives, alkenyl substituted sirolimus derivatives, 32-O-dihydro or 32-O-substituted sirolimus derivatives, 32-deoxorapamycin, and 16-pent-2-ynyloxy-32(S)-dihydrorapamycin.

4. The process according to claim 3, wherein the sirolimus derivative is one or more selected from the group consisting of sirolimus and everolimus.

5. The process according to claim 1, wherein the water-soluble carrier is one or more selected from the group consisting of hydroxy propyl methylcellulose (HPMC), hydroxy propyl methylcellulose phthalate, polyvinyl pyrrolidone (PVP), hydroxypropyl cellulose (HPC) or its derivatives, polyethylene glycol (PEG), saturated polyglycolised glycerides, cycicodextrines, polyvinyl alcohol, polyethylene oxide, polyethylene glycol, hydroxypropyl cellulose, hydroxyethyl cellulose, vinyl pyrrolidone-vinyl acetate copolymer, arginate, calcium carboxymethyl cellulose, sodium carboxymethyl cellulose, carbomer, carrageenan, chitosan, guar gum, and dimethyl aminoethyl methacrylate-buthyl methacrylate-methyl methacrylate copolymer.

6. The process according to claim 1, wherein the organic solvent is one or more selected from the group consisting of a linear or branched alcohol having 1 to 10 carbon atoms, an ester having 3 to 10 carbon atoms, a polar or non-polar ether having 3 to 10 carbon atoms, a polar or non-polar ketone having 1 to 10 carbon atoms, and a halogenated hydrocarbon having 1 to 10 carbon atoms.

7. The process according to claim 6, wherein the organic solvent is one or more selected from the group consisting of a linear or branched alcohol having 1 to 5 carbon atoms, an ester having 3 to 6 carbon atoms, a polar or non-polar ether having 3 to 6 carbon atoms, a polar or non-polar ketone having 1 to 5 carbon atoms, and a halogenated hydrocarbon having 1 to 5 carbon atoms

8. The process according to claim 1, wherein the solvent is used in the amount of 0.05 to 500 ml per 1 g of the sirolimus derivative.

9. The process according to claim 1, wherein the ratio between the used amounts of the solution of the sirolimus derivative and the water-soluble carrier is 1:0.05 to 1:500 based on the weight.

10. A formulation of a sirolimus derivative prepared from the preparation process of claim 1, wherein
the content of the sirolimus derivative is 0.01 to 40% by weight based on the total weight of the formulation;
the content of the sirolimus derivative after a 60 hour storage at 80° C. is no less than 80% by weight with respect to the initial content; and
it is in the form of granules having a uniform average particle size.

11. The formulation of a sirolimus derivative of claim 10, wherein the average particle size is 0.01 to 500 μm.
